(19) United States
(12) Patent Application Publication
(10) Pub. No.: US 2007/0004916 A1
(43) Pub. Date: Jan. 4, 2007

(54) Process for the production of cefotaxime sodium

(75) Inventors: Siddiqui Mohammed Jaweed Mukarram, Aurangabad (IN); Rashid Abdul Rehman Khan, Aurangabad (IN); Ram Prasad Yadav, Aurangabad (IN); Mohammed Younus Khan, Aurangabad (IN)

Correspondence Address: IPLM GROUP, P.A., POST OFFICE BOX 18455, MINNEAPOLIS, MN, 55418, US

(73) Assignee: Wockhardt Limited

(21) Appl. No.: 11/487,959

(22) Filed: Jul. 17, 2006

(30) Foreign Application Priority Data
Jan. 16, 2004 (WO) PCT/IB04/00090

(51) Int. Cl. C07D 501/14

(52) U.S. Cl. 540/228

(57) ABSTRACT

A process for the production of 7-[2-(2-amino-4-thiazolyl)-2-syn-methoxyimino-acetamido]-3-acetoxymethyl-3-cephem-4-carboxylic acid (Cefotaxime) in aqueous isopropyl alcohol is provided. The synthesis provides the product in greater than 99 % HPLC purity.

### CROSS-REFERENCE TO RELATED APPLICATION

This application claims priority from international patent application Serial No. PCT/IB2004/000090 filed Jan. 16, 2004, and published in English on Aug. 25, 2005 as International Publication No. WO 2005/076694 A2, which are incorporated herein by reference.

### FIELD OF THE INVENTION

The present invention relates to an improved process for the production of 7-[2-(2-aminpthiazol-4-yl)-2-syn-methoxyiminoacetamido]-3-acetoxymethyl-3-cephem-4-carboxylic acid (Cefotaxime) and its sodium salt. The synthesis of Cefotaxime includes the reaction of 2-(2-chloroacetamidothiazol-4-yl)-2-syn-methoxyiminoacetyl chloride with 7-aminocephalo-sporanic acid (7-ACA) in a mixture of isopropyl alcohol and water. The amino protected Cefotaxime is subsequently de-protected using thiourea and a mild base in aqueous isopropyl alcohol to obtain Cefotaxime acid. The acid is converted into the sodium salt using sodium-2-ethylhexanoate in of ethyl acetate, methanol and triethylamine to obtain Cefotaxime sodium in greater than 99% HPLC purity.

### BACKGROUND OF THE INVENTION

Cephalosporin antibiotics inhibit bacteria by interfering with the synthesis of essential structural components of the bacterial cell wall. They are considered as highly effective antibiotics with low toxicity and can be used for treatment of a wide variety of bacterial infections. A number of cephalosporin derivatives have been discovered with increased potency and improved stability. Ochiai et al. (U.S. Pat. No. 4,098,888) disclose cephem compounds and processes for their preparation. Heymes et al. (U.S. Pat. No.4,152,432) disclose 3-acetoxymethyl-7-(iminoacetamido)cephalosporonic acid derivatives, in particular cefotaxime, and process for preparing the derivatives.

7-ACA (7-Amino-3-acetoxymethyl-3-cephem-4-carboxylic acid) having the formula IA: 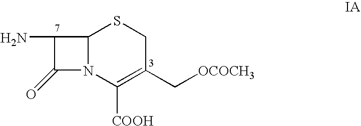
is known, and has been proposed as starting material in various syntheses, in particular in the synthesis of cephalosporins. Various cephalosporins can be obtained through the following reaction steps;
1. acylation of the 7-amino group of the cephalosporanic ring with an optionally substituted aminothiazolyl acetic acid wherein the amino group has been protected;
2. de-protecting the amino protecting group; and
3. optionally converting the 3-acetoxymethyl group of the cephalosporanic ring to another group using a nucleophilic agent.

The order of these steps may be varied as desired. Typically, the acylation of the 7-amino group of the cephalosporanic ring is carried out with an optionally substituted aminothiazolyl acetic acid whose amino group has been protected, followed by the de-protection of the amino group.

U.S. Pat. No. 4,767,852 discloses a process for the preparation of known 2-oxyiminoacetamido-3-cephem-4-carboxylic acid derivatives, including cefotaxime and ceftriaxone, by acylating 7-amino-3-cephem-4-carboxylic acid derivatives already substituted at the 3-position with 2-mercaptobenzothiazolyl-(Z)-2-(2-aminothiazol-4-yl)-2-methoxyiminoacetate, the latter being often referred to as MAEM. Similarly, U.S. Pat. No. 5,026,843 discloses a process for preparing ceftriaxone disodium salt hemi-heptahydrate. As the first step in the process disclosed in that patent, 7-amino-cephalosporanic acid (7-ACA) already suitably substituted at the 3-position is acylated at the 7-position using MAEM as the acylating agent. MAEM has become the standard acylating agent for the preparation of cephalosporins having an oximino group and a 2-aminothiazolyl group in the 7-acylamido side chain. However, a byproduct of this reaction is the toxic compound, viz., 2-mercaptobenzothiazole.

U.S. Pat. No. 5,317,099 discloses a process for the synthesis of β-lactam derivatives such as cefotaxime and ceftriaxone in which silylated 7-ACA is acylated with acyloxyphosphonium chloride derivative of 2-(2-aminothiazol-4-yl)-2-syn-methoxyimino acetic acid, which in turn is prepared from triphenylphosphine (TPP), hexachloroethane or carbon tetrachloride and 2-(2-aminothiazol-4-yl)-2-syn-methoxyimino acetic acid. However, the use of TPP as a reactant can increase the overall cost to prepare the cefotaxime.

U.S. Pat. No. 5,037,988 discloses a process for the production of cephalosporins, in particular cefotaxime and ceftrioxane, in which an activated form of an organic acid, i.e., 2-(2-aminothiazol-4-yl)-2-oxyiminoacetyl sulfitedialkyl-formiminium halide hydrohalide having Formula A is coupled with a 7-aminocephalosporanic acid derivative. 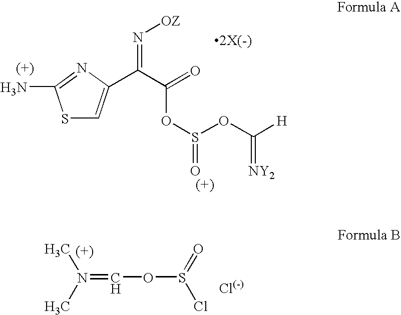
The compound of Formula A was prepared by reacting 2-(2-aminothiazol-4-yl)-2-oximnino acetic acid with dimethylformiminium chloride chlorosulfite of Formula B, which in turn was prepared by reacting approximately equimolar quantities of thionyl chloride and dimethylformamide at room temperature in specific solvents only like benzene or toluene.

U.S. Pat. No. 5,654,425 discloses a method for acylation of the 7-amino group of the cephalosporanic ring, according to which a 7-ACA aminothiazolyl protected adduct is prepared by acylating,the amino group with an aminothiazolyl acetic acid having the amino group protected with a phenyl acetyl or a phenoxy acetyl group, the amino group can be de-protected with aqueous hydrolysis in the presence of penicillin G amidase or penicillin V amidase, respectively.

Thus, there is a need for an efficient and inexpensive synthesis of Cefotaxime having high purity.

### SUMMARY OF THE INVENTION

The present invention provides a process for the preparing and isolating substantially pure sodium, 7-[2-(2-aminothiazol-4-yl)-2-syn-methoxyiminoacetamido]-3-acetoxymethyl-3-cephem-4-carboxylate, having Formula VII

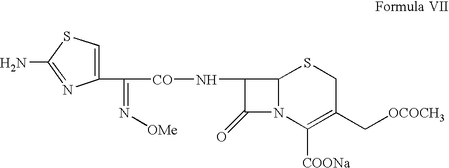

In one embodiment, a process is provided for the production of Cefotaxime and the sodium salt of Cefotaxime. The process includes a reaction of 2-(2-chloroaceta-midothiazol-4-yl)-2-syn-methoxyiminoacetyl chloride with 7-aminocephalosporanic acid (7-ACA) in aqueous isopropyl alcohol to obtain an amino protected Cefotaxime having Formula V:

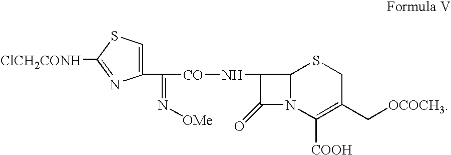

The chloroacetyl group of Formula V compound is de-protected using thiourea and a mild base in a mixture of water and isopropyl alcohol. The pH of the reaction mixture is raised to about 3.0 to obtain a white precipitate of Cefotaxime with high purity. The sodium salt of Cefotaxime is obtained by reacting the sodium-2-ethylhexanoate in the presence of triethylamine and a mixture of organic solvents. Finally, Sodium, 7-[2-(2-aminothiazol-4-yl)-2-syn-methoxyiminoacetamido]-3-acetoxymethyl-3-cephem-4-carboxylate is obtained in more than 99% HPLC pure form without any unknown impurity in more than 0.1%.

These and other aspects of the invention will be apparent from the accompanying specification. In no event, however, should the above summaries or the terminology employed for the purpose of describing particular embodiments be construed as limitations on the claimed subject matter, which subject matter is defined solely by the attached claims, as may be amended during prosecution.

### DETAILED DESCRIPTION OF THE INVENTION

The present invention provides a simple and efficient process for the preparation of a cephalosporin, e.g., Cefotaxime Sodium (Formula VII). The process of invention uses inexpensive and readily available starting materials, short reaction times, and simple isolation processes to provide Cefotaxime Sodium in excellent purity.

The process of present invention comprises protection of the exocyclic amino unctional group of 2-(2-aminothiazol-4-yl)-2-syn-methoxyiminoacetic acid (Formula I) with chloroacetyl chloride in N,N-dimethylacetamide as shown in the reaction scheme below:

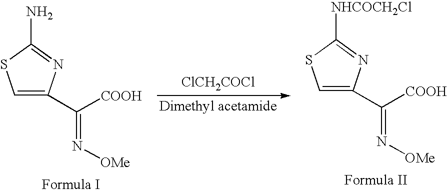

The chloroacetyl chloride is added at a low temperature, with stirring. After the addition is complete the temperature is allowed to rise to 30 to 35° C. and stirring is continued until the amino group is completely protected. The crude reaction mixture is poured into water to precipitate out the 2-(2-chloroacetylaminothiazol-4-yl)-2-syn-methoxyiminoacetic acid (Formula II). The precipitate of the title compound is obtained, after filtration and vacuum drying, in quantitative yield.

The carboxy group of 2-(2-chloroacetylaminothiazol-4-yl)-2-syn-methoxy-iminoacetic acid is converted to an acid chloride by reacting the acid with phosphorous pentachloride at a temperature of from about −10 to about 25° C., more preferably the reaction temperature is from about −5 to about 10° C. 2-(2-Chloroacetylaminothiazol-4-yl)-2-syn-methoxyiminoacetyl chloride (Formula III); 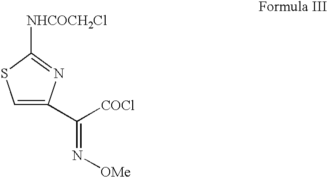
is obtained as solid after filtration and vacuum drying. The product obtained is used for acylation in the next step, without any further purification.

The synthesis of 7-[2-(2-Chloroacetamidothiazol-4-yl)-2-syn-methoxyimino)-acetamido]-3-acetoxymethyl-3-cephem-4-carboxylic acid (N-Chloroacetamido cefotaxime, (Formula V); 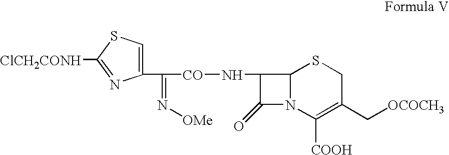
is carried out by acylating 7-Amino-3-acetoxymethyl-3-cephem-4-carboxylic acid, (Formula IV); 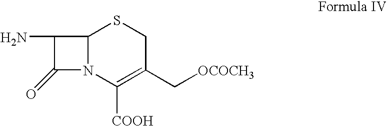
with 2-(2-chloroacetylaminothiazol-4-yl)-2-syn-methoxyiminoacetyl chloride (Formula III) in aqueous isopropanol, in the presence of a base. Examples of suitable bases for this reaction include alkali metal carbonate or alkali metal hydroxide. Examples of suitable bases include sodium carbonate and sodium hydroxide. The acylation reaction is maintained at a temperature of from about −10 to about 30° C. Preferably, the reaction temperature is from about −5 to about 10° C. The acylation process is usually complete within about 15 to 20 minutes. After the acylation reaction is complete, the pH of the reaction mixture is adjusted to about 2.0 to about 4.0, more preferrably, from about.2.5 to about 3.0, using dilute hydrochloric acid solution to obtain a precipitate of the N-chloroacetamido cefotaxime acid (Formula V). The precipitate obtained from the reaction is isolated by filtration.

In another embodiment, the amino group of 7-[2-(2-chloroacetamidothiazol-4-yl)-2-syn-methoxyiminoacetamido]-3-acetoxymethyl-3-cephem-4-carboxylic acid is de-protected. The de-protection of the amino group is carried out in water or a mixture of water and alcohol. Alcohols suitable for this reaction include methanol, ethanol or isopropanol. More particularly, a mixture of water and isopropanol is preferred as solvent. The chloroacetyl group from Formula V is removed in the presence of thiourea at a pH of from about 5.0 to about 8.0, more preferably, of from about 6.5 to about 7.5. The pH is adjusted using a base selected from the group consisting of alkali metal carbonates or alkali metal hydroxides. The reaction is carried out at temperature of from about 10° C. to about 40° C., preferably at a temperature of from about 20° C. to 30° C. The reaction usually is complete in about 6 to 8 hours. The pH of the reaction mixture is adjusted to about 2.0 to about 4.0, most preferably, from about 2.7 to about 3.0 to obtain a precipitate of Cefotaxime acid having Formula VI:

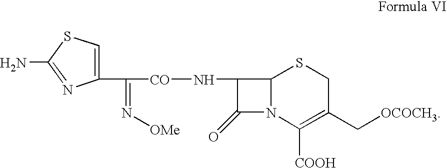

Cefotaxime acid is converted to cefotaxime sodium (Formula VII); 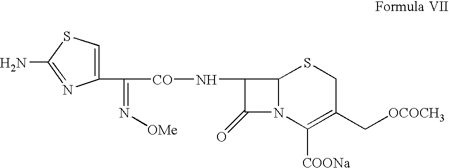
in a mixture of methanol and ethyl acetate in presence of triethylamine and sodium-2-ethylhexanonate. The product is precipitated by addition of excess ethyl acetate which on filtration provides final product (Formula VII) as solid mass. The Cefotaxime Sodium prepared by the process of the invention is obtained in good yield and high purity.

The purity of the Cefotaxime Sodium obtained by the above process is greater than 99% (HPLC assessment) and no unknown impurity is observed in greater than 0.10%. The process repeatedly provides a product with absorbance value not greater than 0.07 at 430 nm.

### EXAMPLES

The following examples illustrate the invention, but is not limiting thereof,

### Example 1

### 7-[2-(2-Chloroacetamidothiazol-4-yl)-2-syn-methoxyimino-acetamido]-3-acetoxymethyl-3-cephem-4-carboxylic acid (N-Chloroacetamido cefotaxime, V)

### Step I: 2-(2-Chloroacetamidothiazol-4-yl)-2-syn-methoxyiminoacetic acid (II)

Chloroacetyl chloride (56.2 g) is added to a solution of 2-(2-aminothiazol-4-yl)-2-syn-methoxyiminoacetic acid (Formula I, 100 g) and 1000 ml of N,N-dimethyl acetamide at temperature of from −5° C. to 5° C. The temperature of the reaction mixture is gradually increased to from 30° C. to 35° C. and stirred until the disappearance of the starting material. After the reaction is complete, the mixture is poured into 1000 ml of cold water at 5° C. and stirred allow the product to precipitate. The precipitate obtained is filtered, washed with water, and dried under vacuum to provide 2-(2-chloro-acetamidothiazol-4-yl)-2-syn-methoxyiminoacetic acid (Formula II).

### Step II: 2-(2-Chloroacetamidothiazol-4-yl)-2-syn-methoxyiminoacetyl chloride (III)

Phosphorus pentachloride (106 g) is added in portions to a stirred solution of 2-(2-chloroacetamidothiazol-4-yl)-2-syn-methoxyiminoacetic acid (Formula II, 137 g) and 1500 ml dichloromethane at temperature of from −5 to 0° C. under nitrogen atmosphere. The reaction mixture is stirred at 0° C. for 90 minutes to obtain the acid chloride of 2-(2-chloroacetamido-thiazol-4-yl)-2-syn-methoxyimino acetic acid as a precipitate. The product obtained is used in the next step without any additional purification.

### Step III: 7-[2-(2-Chloroacetamidothiazol-4-yl)-2-syn-methoxyiminoacetamido]-3-acetoxymethyl-3-cephem-4-carboxylic acid (N-Chloroacetamido cefotaxime, V)

The 2-(2-chloroacetamidothiazol-4-yl)-2-syn-methoxyiminoacetyl chloride (Formula III), obtained in Step II, is added portion-wise to a mixture of 7-amino-cephalosporanic acid (100 g), 400 ml water, 400 ml isopropanol and sodium carbonate (27 g). The pH of the reaction mixture is maintained at 6.5 to 7.5 by addition of sodium carbonate solution, and at a temperature of from −5 to 5° C. The progress of the acylation reaction is monitored by HPLC. After disappearance of starting material, the pH of the solution is adjusted to about 2.7 to 3.0 using dilute hydrochloric acid. This provides the reaction mixture in the form of a slurry, which is filtered and washed with water. The wet product is used as is in the next step, i.e., de-protection of the amino function, without any additional purification.

### Example 2

### 7-[2-(2-Aminothiazol-4-yl)-2-syn-methoxyiminoacetamido]-3-acetoxymethyl-3-cephem-4-carboxylic acid (Cefotaxime acid, VI)

7-[2-(2-Chloroacetamidothiazol-4-yl)-2-syn-methoxyiminoacetamido]-3-acetoxy-methyl-3-cephem-4-carboxylic acid (Formula V) (wet product from Step III of Example 1), thiourea (50 g) and sodium carbonate (40 g) are suspended in a mixture of 200 ml water and 400 ml of isopropyl alcohol at a temperature of from 20° C. to 30° C. Sodium carbonate is added to the reaction mixture to obtain a clear solution. The progress of the de-protection of the chloroacetyl group is monitored by HPLC. After completion of the de-protection reaction, the crude solution is decolorized with active charcoal and filtered. The pH of the filtrate is adjusted to from 2.7 to 3.0 with dilute hydrochloric acid, at temperature of from 20° C. and 30° C. to provide a precipitate of Cefotaxime. The reaction mixture is stirred for an additional 2 hours, filtered, washed with isopropyl alcohol, and dried under vacuum to provide white color Cefotaxime acid (Formula VI) in high purity.

### Example 3

### Sodium, 7-[2-(2-aminothiazol-4-yl)-2-syn-methoxyimino-acetamido]-3-acetoxymethyl-3-cephem-4-carboxylate (Cefotaxime sodium, VII)

Cefotaxime acid (Formula VI, 100 g) prepared in Example 2 is suspended in a mixture of 300 ml methanol and 200 ml ethyl acetate followed by addition of triethyl-amine (28.8 g) at a temperature of from −5° C. to 5° C. The solution obtained is treated with activated charcoal (10 g) and filtered. A solution of sodium-2-ethylhexanoate (60 g) in 400 ml ethyl acetate is added to the colorless filtrate at a temperature of from −5 to 5° C. The Cefotaxime sodium is precipitated by diluting the reaction mixture with additional ethyl acetate. The slurry containing the Cefotaxime sodium is filtered, washed with cold ethyl acetate and dried under vacuum to obtain a white material having HPLC purity more than 99%, without any impurity greater than 0.1%.

All references cited herein are expressly incorporated herein by reference in their entirety into this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure and the claims shown below are not limited to the illustrative embodiments set forth herein.

We claim:

1. A process for preparing a compound of Formula (VII) comprising: 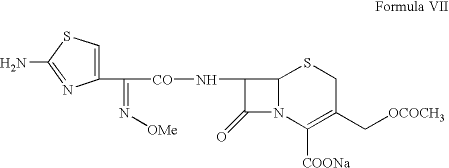
(a) contacting a compound of the Formula IV 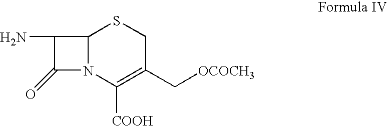
with 2-(2-Chloroacetamidothiazol-4-yl)-2-syn-methoxyiminoacetyl chloride and a base, in a solvent; to provide 7-[2-(2-chloroacetamidothiazol-4-yl)-2-syn-methoxyiminoacetamido]-3-acetoxymethyl-3-cephem-4-carboxylic acid;
(b) converting 7-[2-(2-chloroacetamidothiazol-4-yl-2-syn-methoxyiminoacetamido]-3-acetoxymethyl-3-cephem-4-carboxylic acid into 7-[2-(2-aminothiazol-4-yl)-2-syn-methoxyiminoacetamido]-3-acetoxymethyl-3-cephem-4-carboxylic acid in presence of a solvent and a de-protecting protecting agent; and
(c) isolating the 7-[2-(2-aminothiazol-4-yl)-2-syn-methoxyiminoacetamido]-3-acetoxymethyl-3-cephem-4-carboxylic acid.

2. The process of claim 1, wherein the 7-[2-(2-aminothiazol-4-yl)-2-syn-methoxyiminoacetamido]-3-acetoxymethyl-3-cephem-4-carboxylic is converted into sodium, 7-[2-(2-aminothiazol-4-yl)-2-syn-methoxyiminoacetamido]-3-acetoxymethyl-3-cephem-4-carboxylate.

3. The process of claim 1, wherein the solvent in step (a) comprises an aliphatic alcohol, water, or a mixture thereof.

4. The process of claim 3, wherein the aliphatic alcohol comprises isopropyl alcohol.

5. The process of claim 3, wherein solvent comprises a mixture of isopropyl alcohol and water.

6. The process of claim 5, wherein volume ratio of isopropyl alcohol and water is about 1 to 1.

7. The process of claim 1, wherein 7-Amino-3-acetoxymethyl-3-cephem-4-carboxylic acid (Formula IV) in step (a) is dissolved in aqueous isopropyl alcohol and an alkali metal carbonate or alkali metal hydroxide prior to addition of 2-(2-Chloroacetamido-thiazol-4-yl)-2-syn-methoxyiminoacetyl chloride.

8. The process of claim 7, wherein the alkali metal carbonate is sodium carbonate.

9. The process of claim 1, wherein pH of the mixture formed in step (a) is between about 6.0 to about 8.0.

10. The process of claim 1, wherein the contact time for step (a) is between about 10 min to about 2 hours.

11. The process of claim 1, wherein the contact temperature for step (a) is between about −5 to about 20° C.

12. The process of claim 1, wherein pH of the mixture formed in step (b) is adjusted to about 2 to about 4 using mineral acid, after the reaction is complete.

13. The process of claim 12, wherein the mineral acid is dilute hydrochloric acid.

14. The process of claim 13, wherein reaction mixture is stirred for 2 hours to obtain a slurry.

15. The process of claim 12-14, wherein the slurry is filtered and washed with water and isopropyl ether to provide 7-[2-(2-chloroacetamido-thiazol-4-yl)-2-syn-methoxyimino-acetamido]-3-acetoxymethyl-3-cephem-4-carboxylic acid.

16. The process of claim 1, wherein step (c) is carried out in aqueous alcohol.

17. The process of claim 16, wherein volume ratio of isopropyl alcohol and water is about 2 to 1.

18. The process of claim 1 (c), wherein thiourea is added to 7-[2-(2-chloro-acetamido-thiazol-4-yl)-2-syn-methoxyiminoacetamido]-3-acetoxymethyl-3-cephem-4-carboxylic acid followed by addition of sodium carbonate to provide a clear solution.

19. The process of claim 1, wherein reaction mixture in step (c) is stirred for about 5 to about 10 hours.

20. The process of claim 1, wherein the reaction in step (c) is conducted at a temperature of from about 10 to about 40° C.

21. There process of claim 1, wherein the pH of the reaction mixture in step (d) is adjusted to about 2.0 to about 4.0 using concentrated hydrochloric acid.

22. The process of claim 21, wherein the reaction mixture is stirred for about 2 hours to provide a slurry.

23. The process of claim 22, wherein the slurry is filtered and washed with water and isopropyl ether to provide 7-[2-(2-aminothiazol-4-yl)-2-syn-methoxyiminoacetamido]-3-acetoxymethyl-3-cephem-4-carboxylic acid.

24. The process of claim 2, wherein the acid is contacted with sodium-2-ethylhexanoate in a suitable solvent or solvent mixture, and a base.

25. The process of claim 24, wherein the solvent comprises ethyl acetate and the solvent mixture comprises ethyl acetate and ethanol.

26. The process of claim 24, wherein the base is triethylamine.

27. The process of claim 24, wherein excess of ethyl acetate is added to get the precipitate the sodium, 7-[2-(2-aminothiazol-4-yl)-2-syn-methoxyimino-acetamido]-3-acetoxymethyl-3-cephem-4-carboxylate after the reaction.

28. The process of claim 1, wherein sodium, 7-[2-(2-aminothiazol-4-yl)-2-syn-methoxyiminoacetamido]-3-acetoxymethyl-3-cephem-4-carboxylic acid is obtained in more than 99% HPLC purity.

29. A process for the preparing a compound of Formula (II) 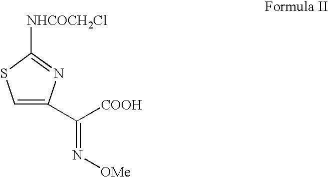
comprising:
(a) contacting a compound of the Formula (I) 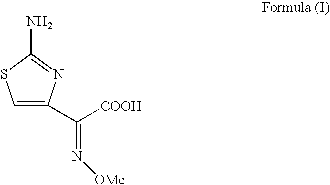
with chloroacetyl chloride in presence of a solvent for a sufficient time to form 2-(2-Chloroacetylthiazolyl-4-yl-2-syn-methoxyiminoacetic acid; and
(b) isolating the 2-(2-Chloroacetylthiazolyl-4-yl-2-syn-methoxyimino acetic acid.

30. The process of claim 29, wherein the solvent in step (a) is an aliphatic amide.

31. The process of claim 30, wherein the aliphatic amide is N,N-dimethylformamide or N,N-dimethylacetamide.

32. The process of claim 31, wherein more preferable solvent is N,N-dimethylactamide.

33. The process of claim 29, wherein chloroacetylchloride in step (a) is added below 5° C.

34. The process of claim 29, wherein reaction temperature in step (a) is from about 20 to 40° C.

35. The process of claim 29, wherein reaction mixture in step (a) is stirred for about 2 to about 6 hours.

36. The process of claim 29, wherein water is added to the reaction mixture in step (b) to precipitate the 2-(2-Chloroacetylthiazolyl-4-yl-2-syn-methoxyiminoacetic acid, after the amino protection reaction is complete.

37. The process of claim 36, wherein 2-(2-Chloroacetylthiazolyl-4-yl-2-syn-methoxyiminoacetic acid is filtered and dried to provide a dry powder.

38. A process for the manufacturing a compound of Formula (III) 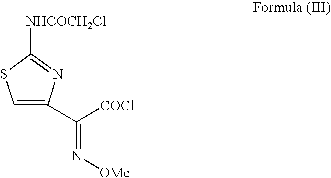
comprising:
(a) contacting 2-(2-Chloroacetylthiazolyl-4-yl-2-syn-methoxyiminoacetic acid with phosphorous pentachloride in suitable solvent; and
(b) isolation of 2-(2-Chloroacetylthiazolyl-4-yl-2-syn-methoxyiminoacetyl chloride.

39. The process of claim 38, wherein step (a) is carried out in dichloromethane.

40. The process of claim 38, wherein phosphorous pentachloride is mixed with dichloromethane in step (a) and added below 0° C. to 2-(2-chloroacetylthiazolyl-4-yl-2-syn-methoxyimino acetic acid.

41. The process of claim 38, wherein contact time in step (a) is between about 30 min to about 2 hours.

42. The process of claim 38, wherein 2-(2-chloroacetylthiazolyl-4-yl-2-syn-methoxyiminoacetyl chloride is filtered and washed with isopropyl ether in step (b) to get solid mass, which itself is used for further step.
